(19) United States
(12) Patent Application Publication
(10) Pub. No.: US 2026/0284937 A1
(43) Pub. Date: Sep. 24, 2026

(54) KIT FOR A FORMING PROCESS OF COSMETIC PRODUCTS, FORMING PROCESS AND PRODUCTS OBTAINED

(71) Applicant: GOTHA COSMETICS S.R.L., LALLIO (BG) (IT)

(72) Inventor: Luca DALLATORRE, LALLIO (IT)

(21) Appl. No.: 19/478,926

(22) PCT Filed: Apr. 24, 2024
(86) PCT No.: PCT/IB2024/054006

§ 371 Date: Oct. 27, 2025

(30) Foreign Application Priority Data
Apr. 27, 2023 (IT) 102023000008325

(51) Int. Cl. B29C 39/38; B29C 39/02; B29C 39/26; B29L 31/00

(52) U.S. Cl. CPC B29C 39/38; B29C 39/02; B29C 39/26; B29L 2031/718

(57) ABSTRACT

The disclosure relates to a kit comprising a mould having a chamber provided with an access opening and a support element for a moulding process of a cosmetic product P in semi-solid or paste form, an article comprising the cosmetic product P and said support element and a moulding process of said cosmetic product P.

### BACKGROUND

The present invention concerns a moulding and packaging process of cosmetic products in paste, semi-solid or gel form. In particular, the invention relates to a moulding and packaging process that allows the cosmetic product to be obtained with a desired shape and, at the same time, also coupled to a support element (also called pan), the latter being adapted to be fixed directly in a primary container.

The invention also concerns the cosmetic product fixed to the support element, and the support element per se used in this process and a kit comprising a mould and said support element used in this process.

Cosmetic products of this type belong to the following categories: foundations, eye shadows, blushers, bronzers, highlighters, lipsticks or the like. The cosmetic products concerned by this application have the additional feature of being in solid, semi-solid, paste or gel form.

With the exception of lipsticks, said products are typically in pod form and hereinafter in this description will be identified, as a whole, as cast products or, more specifically, poured products.

According to a common prior art, these cosmetic products are obtained by means of a moulding process by pouring into a support, the latter being in the form of a receptacle with a wide opening. The process makes use of mechanical, or volumetric, dosing devices, which dose the semi-finished cosmetic product poured inside the receptacle from said opening in a suitable quantity in relation to the filling volume desired for the receptacle into which the cosmetic product flows. The cosmetic product is, at this stage, mainly semi-fluid and therefore is evenly distributed inside the receptacle, in which it will be cooled to solid state. The cosmetic product will thus have a flat surface at the opening of the receptacle. Subsequently, the solid cosmetic product contained in said receptacle will be subjected to packaging. After the packaging is opened, the cosmetic product will present this flat surface to the consumer.

The drawback of this process is that it is not possible to give said surface of the cosmetic product displayed to the consumer a three-dimensional shape that represents, for example, a brand, a pattern or the like.

According to another moulding technique of a cosmetic product, known as “back pouring” and described in the patent application EP 3669700 A1, the moulding process involves the use of a mould comprising at least one hollow body having a hollow body delimited by a wall with a moulding surface and an access opening delimitated by an edge. The latter is placed on the back of the mould. The mould further comprises a multifunctional element delimited by an edge. This multifunctional element is a closing element as it acts as closure of the opening of the hollow body; this closing element is positioned at the same level as the edge of the opening. In other words there is no overlapping between the closing element and the edge of the opening. This multifunctional element also acts as support element of the cosmetic product once packaged.

The body is made of a flexible polymeric material. The closing element is made of plastic material, such as acrylonitrile-butadiene-styrene (ABS) copolymer or polymers with equivalent mechanical properties.

The hollow body is adapted to receive the cosmetic product in liquid, or at least semi-liquid, state.

In this process, before filling the hollow body with the cosmetic product in said state, the hollow body is closed by placing the closing element on its access opening. The closing element is provided with holes arranged in the central area. The cosmetic product is poured into these holes, which allow the cosmetic product to pass into the hollow body of the mould.

The cosmetic product is then solidified inside the mould into which it was poured. Once the cosmetic product has solidified, the closing element will form a single body with the cast cosmetic product.

Therefore, the use of the mould makes it possible to give the cosmetic product a predetermined desired shape and, for example, to reproduce a brand or a figure on the surface of the cosmetic product that faces the consumer.

The plastic material of which the closing element is made primarily allows a reduction of the weight of the finished packaging and of the costs of packaging materials.

However, a process of this kind has some limitations.

A significant drawback of this process is that the mould is filled using a method that does not allow rapid filling thereof.

Various factors determine the filling speed of the mould.

An important factor is the fluidity of the cosmetic product, as the mould filling operation takes place by percolation of the cosmetic product through the holes of the closing element. In this operation, the flow of the cosmetic product on the closing element, in particular and above all, the wall with the holes, during percolation affects the filling speed. The thickness of the closing element, hence the length of the hole, and the viscosity of the formulation of the chemical product affect the speed of percolation of the cosmetic product into the mould through said holes, percolation which is facilitated and made faster only if the cosmetic product has a relatively low viscosity value when hot. Therefore, to speed up and facilitate said operation, there is the need to operate with a cosmetic product that is as fluid as possible when hot.

Another important factor that influences this speed is the number of holes and the dimension in width of the holes: the lower the number of holes and the smaller they are, the longer it will take to fill the mould.

Moreover, this process requires great precision to position the nozzles that delivery the cosmetic product exactly coinciding with the holes in the closing element.

A further drawback is that of having to use a mechanical or manual method for movement of the multifunctional element, whether considered individually or after being coupled to the cosmetic product.

Yet another drawback concerns the orientation of the pod if it has wording or a pattern, which for aesthetic reasons must be congruent with the pack. Through magnetism it is possible to orient the product without touching it and spoiling the surface.

Moreover, in general, processes of the prior art use moulds with a hollow body having circular opening and a corresponding support element of the same shape as the opening and of a size coinciding with said opening so that it covers the opening completely.

### BRIEF SUMMARY

The Applicant has now surprisingly found an alternative process for moulding a cast or poured cosmetic product P, generally a pod, that overcomes the drawbacks of the aforesaid processes.

A first object of the present invention is to provide a process that can use a wider range of formulations of cosmetic product. In particular, the process according to the present invention allows the use of formulations with a wider hot viscosity range with respect to a process of the prior art in which the cosmetic product must flow through the holes of a support element. In practice, it is possible to use cosmetic products with a formulation with higher viscosity values with respect to the formulations that can be used in said process of the prior art. This is possible due to the fact that the cosmetic product no longer requires to pass through mandatory routes, i.e., the holes of the closing element.

A second object of the present invention is to provide a process that uses a mould that can be filled with a cosmetic product at a greater speed than the process described in the patent application EP 3669700 A1. An evident advantage is that the whole process for moulding and packaging a cosmetic product is faster.

A further object of the present invention is to develop a process that involves the use of a support element and a mould having a hollow body with an opening, and positioning the support element, whose shape and extension not limited by the shape and the extension of said opening, inside the mould. The practical advantage of this is that as the support element has an extension smaller, typically much smaller, than the width of the opening of the hollow body of the mould and a shape that is fully comprised within the opening, it allows the same type of support element to be used for moulds with openings of different width and shape.

Another object of the present invention is that of improving release of the cosmetic product, once solidified, from the mould. As the support element does not rest on the edge of the chamber of the mould it does not create a limitation of physical interference that causes an obstacle during extraction of the solidified cosmetic product from the mould.

Therefore, the object of the present invention is a process that overcomes said drawbacks through the use of a mould with support element according to the present invention, this process, mould and support element being in accordance with the appended claims.

In detail, the process according to the present invention relates to the moulding of a poured cosmetic product P, according to which moulding is obtained by means of a mould provided with an access opening and a support element as described below, this process comprising the following steps:
A) pouring a semi-finished cosmetic product P′ into said mould through said opening;
B) first cooling of the cosmetic product P until its solidification;
C) heating a support element, preferably to a temperature ranging from 50-300° C.;
D) positioning the hot support element on the cosmetic product P at the access opening (3) of the chamber (1b) of the mould;
E) second cooling of the cosmetic product P until solidification of the melted cosmetic product.

Typically, the process according to the present invention further comprising the following steps:
F) applying a layer of adhesive material on the support element; and
G) placing the cosmetic product P in a primary container.

Hereinafter, solidified product or product in solid state or similar means a product that is no longer in a pourable state, but has taken the consistency of the final cosmetic product, typically in paste, semi-solid or gel form.

Moreover, “melted cosmetic product” means a completely melted or even only partially melted or softened product, unless otherwise specified.

Said mould comprises at least one hollow body having a chamber that is (a) delimited by a wall with a moulding surface and (b) provided with an access opening defined by a perimeter.

Generally, a margin extends from the hollow body in a radial direction from said opening.

Said chamber is made of a material adapted to withstand high temperatures, for example from 60 to 120° C., such as those of the hot cosmetic product when it is poured into the chamber of the mould. This material is, for example, a thermoplastic polymer such as PETG, PVC, APET.

Said chamber is of any shape, typically semi-spherical.

Usually and preferably, the chamber has a portion depicting a three-dimensional representation, such as a figure, a logo, a brand, etc. This representation is on the wall or on a wall or, typically, on the bottom of the chamber. As a consequence both of a mould of this type and of the process according to the present invention, the demoulded solid cosmetic product has a face of its surface bearing said three-dimensional representation.

According to the present invention, the support element is in the generic form of a plate provided with a bearing component. Typically but not necessarily, the support element has a circular shape, although other shapes are possible. The description below is provided with reference to the circular shape of said plate.

In detail, the support element comprises a horizontal surface and at least one retaining foot. The at least one retaining foot will rest against the bottom of a primary packaging that will contain the packaged cosmetic product.

This horizontal surface has two opposite faces, differing from each other.

This at least one retaining foot protrudes from one of the two faces of this horizontal surface (hereinafter said face is defined as “retaining face”). Hereinafter, the face opposite the retaining face is defined as “bearing face”. The bearing face will be placed against the semi-finished cosmetic product during the process according to the present invention.

This horizontal surface has any shape, i.e., it can differ from the shape of the access opening. This horizontal surface can have a circular shape, the shape of another geometric figure or an invented shape.

This horizontal surface of the support element has a surface that is significantly smaller, i.e., not exceeding 80%, with respect to that of said access opening, as specified below.

The horizontal surface of the support element has a surface typically ranging from 20 mm² to 13000 mm².

The horizontal surface has through holes.

As mentioned above, said two faces of the horizontal surface of the support element differ from each other. The bearing face is typically flat. Only the retaining face has at least one retaining foot.

The through holes have the following features, taken individually:
they are typically from 5 to 10 in number;
they are preferably equidistant from one another and from the centre of the support element;
they are different from, or typically the same as, each other in shape or size, or both.

These through holes are typically circular in shape. The through hole has a diameter generally ranging from 0.05 to 18 mm, preferably from 2 mm to 15 mm.

The retaining face has at least one first area and one second area, which protrudes with respect to the first area. Typically, the first area surrounds the second area.

In detail, this face comprises a central area and a peripheral area, the latter is a circular crown with respect to the central area, i.e., the peripheral area is arranged around the whole of the edge of this element. Both said areas are flat. The central area is raised with respect to the surrounding peripheral area.

Finally, there is an intermediate area, which joins said peripheral area and said central area. This area is generally in a oblique or even vertical plane.

Said through holes are located in this peripheral area and typically on a same concentric line. This line is preferably in the portion of the peripheral area contiguous to the intermediate area.

As mentioned, the first area is in the central part of the bearing face of the support element.

This central area of the retaining face of the plane forms the retaining foot.

The at least one retaining foot thus has a regular shape, such as a parallelepiped, a cylinder or a truncated cone, the last typically with a circular section. Therefore, this foot has, at a first end, a flat base that acts as bearing surface of the foot. This bearing base will be fixed against the bottom of the primary packaging that will contain the packaged cosmetic product.

This central area, or retaining foot, protrudes, typically by an amount ranging from 0.05 mm to 4.5 mm, preferably from 1 mm to 4 mm, with respect to the surface of the plane of the peripheral area.

In a preferred embodiment of the support element, said at least one retaining foot is in the shape of truncated cone with circular section.

This structure of the support element on the one hand allows the support element to be “anchored” to the cosmetic product and, on the other hand, to be fixed to the packaging by suitable means, as will be explained below.

The support element is made of a metal material, typically selected from aluminium and tin plate.

The semi-finished cosmetic product is hot so as to typically have a semi-fluid or pourable consistency.

The process according to the present invention comprises a step (step A) in which the hot cosmetic product is poured into chamber of the mould through said access opening. The chamber is then filled, partially or completely, with the cosmetic product P′; usually it is filled to the rim of the chamber. The cosmetic product, after being poured into the chamber of the mould, has a free face, also called free surface. In the present description, free face of the cosmetic product means that it is a face of the cosmetic product not in contact with the moulding wall of the chamber of the mould. Hereinafter, the terms “free face” and “free surface” will be used interchangeably.

Indicatively, the bearing face of the support element occupies a surface of the free face of the cosmetic product on which the support element is positioned ranging from 50 to 75% of the entire surface of this free face.

It is evident that said horizontal surface of the support element has dimensions such as to remain completely inserted inside the perimeter of the access opening and without completely closing said access opening, because, as mentioned above, the surface of the horizontal surface of the support element is smaller, indicatively by 25%, than the surface of the cosmetic product, i.e., than the area delimited by the perimeter of the access opening of the chamber of the mould, free surface on which said horizontal surface of the support element rests. It is also evident that the mould used in the process according to the present invention has no means for holding of the support element, nor does the support element comprise means for attachment to the walls of the mould.

After the cosmetic product is in the chamber of the mould, the cosmetic product is subjected to the cooling step (step B).

In the present invention, the cosmetic product is cooled to a solid state. Generally, but not necessarily, the cosmetic product in solid state is in partially crystalline form.

This cooling is carried out until solidification, preferably complete solidification, of the cosmetic product takes place.

Said cooling takes place with any technique.

Generally, cooling methods that allow the solidification process to be made faster are used, such as those that make use of a dynamic blast chiller that operates at temperatures ranging from −20° C. to +15° C.

Subsequently, i.e., only at the end of the solidification step of the cosmetic product (step B), the step of positioning of the support element (step D) is carried out.

In this step D, the support element is placed on the free face of the cosmetic product.

The support element is in a position defined with reference to two parameters.

The first parameter is the “sinking”. The support element is placed on said free face so that said support element is only resting on said free face of the cosmetic product. In other words, the support element preferably has only the bearing face in contact with the cosmetic product. In particular, it is preferable for the retaining face of the support element not to be covered with the cosmetic product. In any case, it is said second area of the retaining face that must not be completely covered by the cosmetic product.

The second parameter is the positioning of the support element with respect to the centre of the free face of the cosmetic product. This positioning is selected preferably so that the support element occupies the central or concentric position with respect to said free face of the cosmetic product.

Before carrying out step (D) of the present process, said step (C) is carried out. In step (C) the support element is heated until it becomes hot enough to melt the solidified cosmetic product, in particular a surface portion of said free face of the solidified cosmetic product. Indicatively, the support element is heated until reaching a temperature ranging from 50 to 300° C.

Various methods of heating of the support element are possible. According to a preferred embodiment, the support element is heated by a heating element, the latter being typically placed in contact with said support element.

According to a preferred embodiment, the support element is heated only on the bearing face.

According to the process of the present invention, once the cosmetic product has solidified, the support element is applied to said free surface of the cosmetic product still contained in the chamber of the mould. Indicatively, step D starts after the cosmetic product has reached room temperature, i.e., around 25° C. Typically, after reaching this temperature, the cosmetic product can be considered a “structured” solid, i.e., anchored to the support.

With this positioning, the bearing face of the support element rests on said free face of the cosmetic product.

The support element, or at least the bearing face of the support element, emits heat. After this support element is placed on the free surface of the cosmetic product, said face and said portion of surface of the cosmetic product come into contact, and at least one portion of this surface is melted by the heat emitted by the bearing face of the support element. Said portion of surface is at least the portion under the bearing face of the support element and, usually, a part around it.

According to a preferred embodiment of this process, the support element is moved by means of an automated movement system. This system is preferably provided with electromagnets.

Depending on whether the electrical circuit that controls operation of the electromagnet is open or closed, the electromagnet respectively attract or repel, in the specific case, the support element.

In particular, with this movement system the support element is moved and placed on the free face of the cosmetic product exactly in the desired position.

The automated system allows fine tuning of the positioning of the support element on said free face of the cosmetic product, with reference to said two parameters.

It is important for the support element to rest on said surface of said free face, as explained above, to prevent the support element from sinking into cosmetic product; this prevents the retaining face of the support element, in particular the second area of said retaining face, from being covered, even only partially, by the cosmetic product.

The subsequent step (step E) to the step of positioning the support element comprises subjecting the cosmetic product once again to cooling (second cooling).

In particular, cooling is carried out until reaching solidification of the melted cosmetic product in the vicinity of the support element, this as only the cosmetic product in the vicinity of the support element is at least partially melted by the heat emitted by the hot support element, after positioning of the hot support element on the solid surface of said free face of the cosmetic product. In practical terms, cooling takes place until the entire mass of the cosmetic product reaches room temperature, i.e., around 25° C. At this temperature the cosmetic product is structured.

Said second cooling allows the melted cosmetic product to cool and to solidify again. In particular, solidification concerns the cosmetic product melted in contact with the hot support element, i.e., which defines and is close to, or under, at least one part of the surface of the free face of the cosmetic product.

This step E starts immediately after the preceding step D, so as to prevent the support element from sinking into the cosmetic product.

This second cooling can be carried out using the same process as the first cooling, i.e., the one used in the previous step B. Also the conditions, mainly the temperature, at which cooling takes place can be the same or can differ, but in any case suitable for the effectiveness of the process. Generally, cooling takes place at room temperature.

At the end of step E, the support element is fixed, or anchored, to said free face of the cosmetic product. In practice, the closing element will form a single body with the cast cosmetic product.

Although the present invention is not limited by any particular operating theory, it is believed that a part of that portion of cosmetic product, which is in fluid or semi-fluid state following contact with the hot bearing face of the support element, can seep along the through holes present in the first area of the support element and thus reach, and be distributed on, the first area of the retaining face of the support element. In this way the cosmetic product forms continuous streams that reach said first area of the retaining face. After the cosmetic product around and inside said holes has solidified, the cosmetic product performs the effect of holding the support element on itself. This holding is of mechanical type.

It must be noted that the retaining face of the support element is structured to allow the cosmetic product to reach, and be distributed in, only the first area (or peripheral area) of the retaining face of the support element.

Due to the convexity of the raised area of the retaining face of the support element, this area is not reached and covered by the cosmetic product.

In the process according to the present invention, the product inside the chamber of the mould, whether or not already in solid state, is released from said chamber.

The step of extraction (step Z) of the cosmetic product in solid state can be carried out before or after applying adhesive material to the feet of the support element. The choice of when to carry out step Z depends on the type of adhesive material applied to the feet of the support element.

After the cosmetic product has been cooled for the second time, and if necessary after the cosmetic product has been extracted from the mould, the process according to the present invention also comprises a further step of applying adhesive material (step F) to the retaining face of the support element. The adhesive material is spread in the form of a thin layer. The adhesive material is applied on the at least one retaining foot of this area, in particular on the bearing base of the retaining foot. The method of applying the adhesive material takes place according to the prior art and thus shall not be described.

If the cosmetic product is still inside the mould at the end of this step F, the cosmetic product is extracted from the mould.

Finally, the cosmetic product fixed to the support element is inserted into a primary container (step H). The adhesive material applied to the retaining face of the support element allows the latter to be attached to the primary container.

After fixing the support element to the primary container, the latter is ready to be sent to the further packaging steps, according to procedures of the prior art.

Another subject matter of the present invention relates to said support element suitable to carry out the process according to the present invention as described above, and to a kit comprising said mould and said support element.

A further object of the present invention relates to an article comprising the cosmetic product and the support element as described above. In particular, the moulded cosmetic product, in solid state, resting on said support element. It is clear that in this article the cosmetic product is no longer contained in the mould.

### BRIEF DESCRIPTION OF THE DRAWINGS

Further features and advantages of the present invention will be more apparent from an example of embodiment as illustrated in the accompanying figures, wherein:

FIG. 1 is a representation in a perspective view from above of an empty mould according to the present invention;

FIGS. 2 and 3 are two perspective views of a support element, respectively from above and from below, according to an embodiment of the present invention;

FIG. 4 is a perspective view from above of a mould filled with cosmetic product and with the support element placed on the cosmetic product; and

FIGS. 5 and 6 are perspective views, respectively from above and from below, of the cosmetic product demoulded from the mould of FIG. 1.

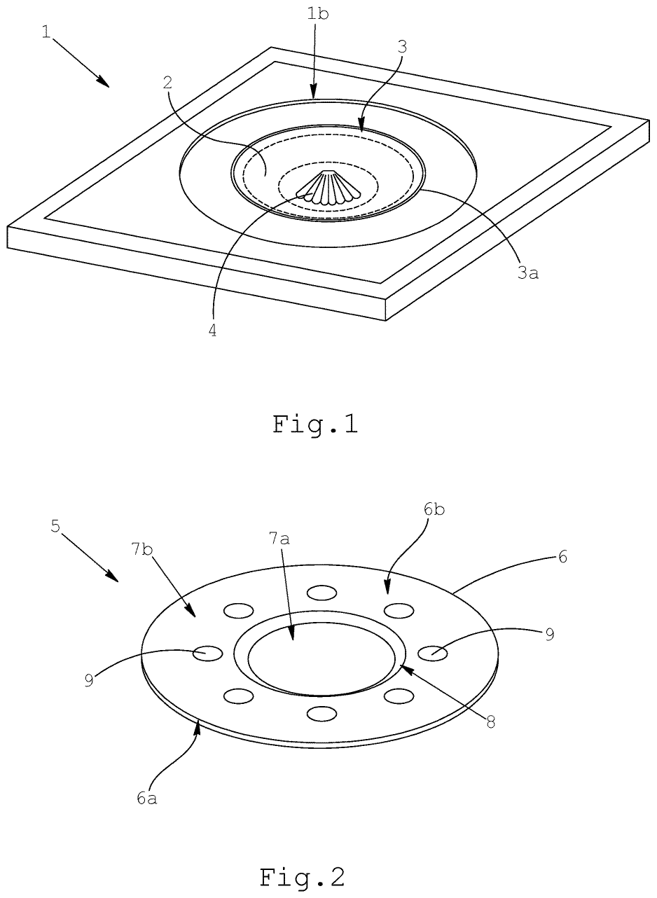

### DETAILED DESCRIPTION

With reference to the accompanying figures, there is illustrated an embodiment of a process according to the present invention in which a thermoformed mould 1 and a support element 5 are used.

The mould 1 comprises a hollow body with a blind bottom. The hollow body forms a chamber 1b having a wall 2 with a moulding surface. In the example of the figures, the chamber 1b has the shape of a spherical cap provided with an access opening 3 defined by a circular perimeter or rim 3a. The wall 2 has a three-dimensional decoration 4 on its bottom, in the case illustrated in the form of a shell.

The access opening 3 has a diameter from 10 mm to 150 mm

The mould 1 is produced by thermomoulding a sheet of thermoplastic polymer, such as PETG, PVC, APET.

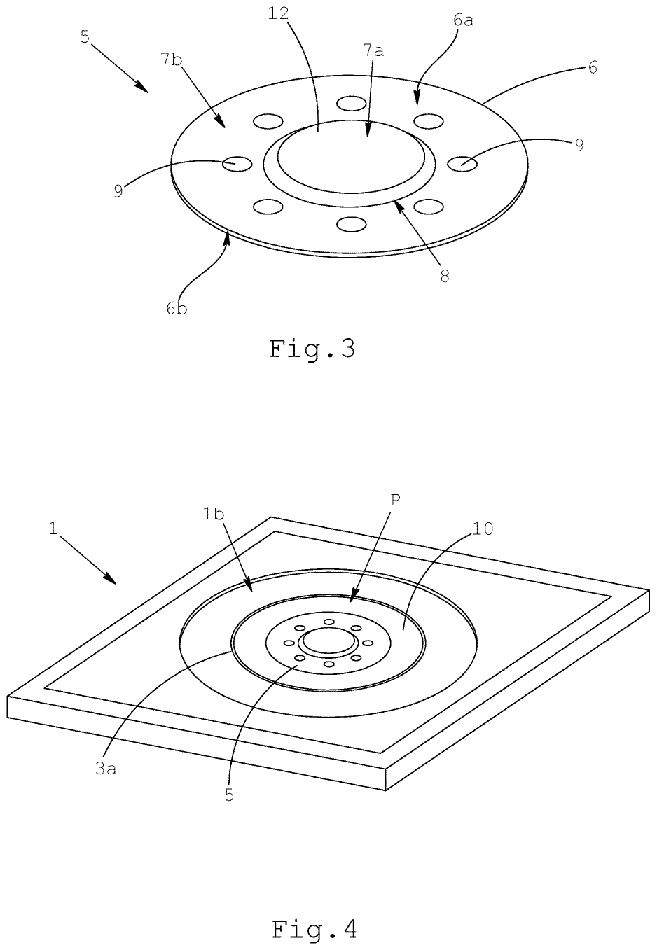

As illustrated in FIGS. 2 and 3, the support element 5 is formed by a plate 6, i.e., a horizontal flat component, and a retaining foot 12.

The plate 6 has a first face (defined here as “retaining face”) 6a with respect to which the retaining foot 12 protrudes and a second face 6b, defined here as “bearing face”. The bearing face will be placed against the semi-finished cosmetic product during the process according to the present invention.

The retaining face 6a has a raised central area 7a, which forms the foot 12, a peripheral area 7b in the form of circular crown, and an intermediate area 8.

The bearing face 6b is flat with a corresponding cavity in the central area.

The peripheral area 7b has 8 through holes 9, the same as, and equidistant from, each other. Each hole has a diameter of 10 mm. The holes are arranged in a circle.

The support element 5 is made of tin plate.

In step (A) of the process exemplified according to the present invention, a semi-finished cosmetic product with a semi-fluid consistency is used.

In step (A) of the process, the cosmetic product P is introduced by pouring, through the access opening 3, into the chamber 1b of said mould 1. The chamber 1b is completely filled with the product P, therefore the product P has a free flat surface 10 at the rim 3a of the chamber 1b.

After the chamber 1b has been filled with the product P, in step (B) the product P is cooled until it has completely solidified. Solidification is obtained by exposing the mould with the product P to room temperature for the time required.

After the product P is solid, the bearing face 6b of the support element 5 is heated (step C) by placing it in contact with a heating element for a time ranging from 0.05 to 45 seconds of this face 6b.

After reaching this temperature of the bearing face 6b, the next step (step D) of the process consists in laying the support element 5 on the surface of the product P that faces the access opening 3. The support element 5 is positioned at the centre of this surface.

The support element 5 is positioned on the free surface 10 of the product P so that only the bearing face 6b of the support element 5 is against said free surface 10 and the retaining face 6a is facing upwards (FIG. 4). The support element 5 is placed carefully to ensure that the retaining face 6a of the support element 5 is not covered by the product P.

At the end of this operation, the next step (step E) consists of once again cooling the mould 1 to room temperature to bring the support element 5, in particular the face 6b, to this temperature and obtain solidification of the portion of product P that was melted following contact with the hot face 6b.

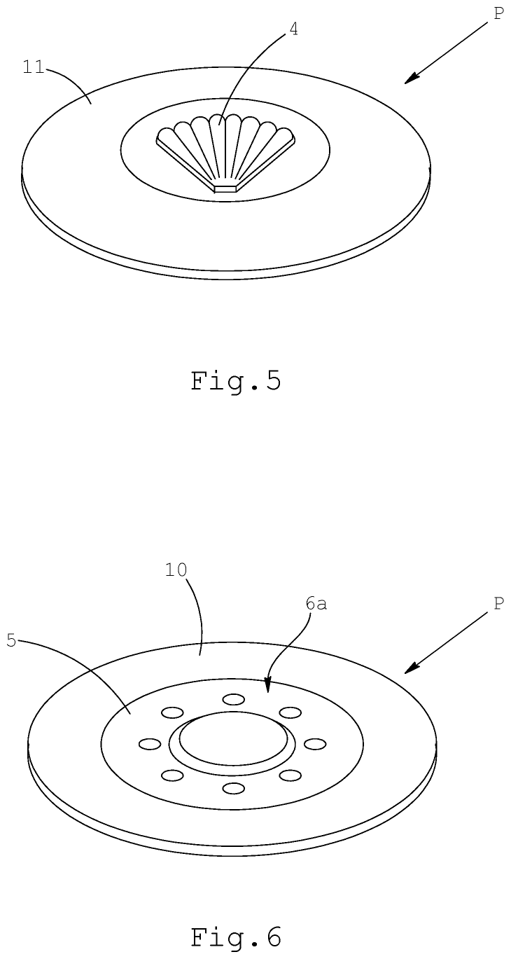

In the next step (step F), when the cosmetic product P and the support element 5 are cold, the cosmetic product P is demoulded and appears as shown in FIGS. 5 and 6.

FIG. 5 shows the convex upper free surface 11 of the solid cosmetic product P after demoulding. Said surface 11 carries the decoration 4.

FIG. 6 shows the free flat surface 10 that carries the support element 5, the retaining face 6a of which is visible.

The further step (step G) is carried out with a dosing machine that places the adhesive material on the base of the retaining foot 12 of the support element 5. The adhesive material forms a thin layer that partially or completely covers the exposed surface of the retaining foot 12.

Finally, the cosmetic product P fixed to the related support element 5 is inserted into the primary packaging which has a bottom on which said retaining foot 12 rests and, by means of said adhesive material, is glued to said retaining foot 12.

Further variants, all deemed to fall within the scope of the invention, can be provided with reference, for example, to the size of, to the number of, and to the dimensions of the through holes and of the cavity in the support element 5.

1.-9. (canceled)

10. A kit for a moulding process of a cosmetic product P in semi-solid or paste form, said kit comprising:
a mould and a support element,
wherein the mould comprises at least one hollow body having a chamber, delimited by a wall with a moulding surface and provided with an access opening defined by a perimeter, and
wherein the support element comprises a horizontal surface and at least one retaining foot, this horizontal surface having a first bearing face and a second retaining face, said at least one retaining foot extending centrally from said retaining face, said horizontal surface having a surface smaller than an area delimited by the perimeter of the access opening of the chamber of the mould.

11. A support element for a kit for moulding a cosmetic product P in semi-solid or past form, the kit including a mould that includes at least one hollow body having a chamber, delimited by a wall with a moulding surface and provided with an access opening defined by a perimeter, the support element comprising:
a horizontal surface and at least one retaining foot, this horizontal surface having a first bearing face and a second retaining face, said at least one retaining foot extending centrally from said retaining face, said horizontal surface having a surface smaller than the area delimited by the perimeter of the access opening of the chamber of the mould.
the second retaining face including a peripheral area surrounding a second central area which, with respect to the peripheral area, is raised, wherein the raised second central area forms the at least one retaining foot.

12. The support element according to claim 11, wherein the peripheral area comprises a circular crown with respect to the central area.

13. The support element according to claim 11, wherein the peripheral area includes through holes.

14. The support element according to claim 11, wherein the support element is made of a metal material.

15. A moulding process of moulding a cosmetic product P in semi-solid or paste form in a mould, wherein the mould is obtained by means of a kit for moulding the cosmetic product P in semi-solid or paste form, wherein the kit includes the mould and a support element according to claim 11, the mould including at least one hollow body having a chamber, delimited by a wall with a moulding surface and provided with an access opening defined by a perimeter, said process comprising steps of:
A) pouring a semi-finished cosmetic product P′ into said chamber of the mould through said access opening;
B) a first cooling of the cosmetic product P′ until the cosmetic product P′ solidifies into the cosmetic product P;
C) heating the support element to a temperature ranging from 50° C. to 300° C.; and
D) positioning the heated support element on the cosmetic product P at the access opening of the chamber of the mould; and
E) a second cooling of the cosmetic product P that has melted until solidification of the melted cosmetic product P.

16. The process according to claim 15, further comprising steps of:
F) applying a layer of adhesive material on the support element; and
G) placing the cosmetic product P in a primary container.

17. The process according to claim 16, wherein the cosmetic product P is extracted from the mould before or after the step F).

18. The support element according to claim 12, wherein said peripheral area has through holes.

19. The support element according to claim 12, wherein said support element is made of a metal material.

20. The support element according to claim 13, wherein said support element is made of a metal material.

21. A moulding process of moulding a cosmetic product P in semi-solid or paste form in a mould, wherein the mould is obtained by means of a kit for moulding the cosmetic product P in semi-solid or paste form, wherein the kit includes the mould and a support element according to claim 12, the mould including at least one hollow body having a chamber, delimited by a wall with a moulding surface and provided with an access opening defined by a perimeter, said process comprising steps of:
A) pouring a semi-finished cosmetic product P′ into said chamber of the mould through said access opening;
B) a first cooling of the cosmetic product P′ until the cosmetic product P′ solidifies into the cosmetic product P;
C) heating a support element to a temperature ranging from 50° C. to 300° C.; and
D) positioning the hot support element on the cosmetic product P at the access opening of the chamber of the mould; and
E) a second cooling of the cosmetic product until solidification of the melted cosmetic product.

22. A moulding process of moulding a cosmetic product P in semi-solid or paste form in a mould, wherein the mould is obtained by means of a kit for moulding the cosmetic product P in semi-solid or paste form, wherein the kit includes the mould and a support element according to claim 13, the mould including at least one hollow body having a chamber, delimited by a wall with a moulding surface and provided with an access opening defined by a perimeter, said process comprising steps of:
A) pouring a semi-finished cosmetic product P′ into said chamber of the mould through said access opening;
B) a first cooling of the cosmetic product P′ until the cosmetic product P′ solidifies into the cosmetic product P;
C) heating a support element to a temperature ranging from 50° C. to 300° C.; and
D) positioning the hot support element on the cosmetic product P at the access opening of the chamber of the mould; and
E) a second cooling of the cosmetic product until solidification of the melted cosmetic product.

23. A moulding process of moulding a cosmetic product P in semi-solid or paste form in a mould, wherein the mould is obtained by means of a kit for moulding the cosmetic product P in semi-solid or paste form, wherein the kit includes the mould and a support element according to claim 14, the mould including at least one hollow body having a chamber, delimited by a wall with a moulding surface and provided with an access opening defined by a perimeter, said process comprising steps of:
A) pouring a semi-finished cosmetic product P′ into said chamber of the mould through said access opening;
B) a first cooling of the cosmetic product P′ until the cosmetic product P′ solidifies into the cosmetic product P;
C) heating a support element to a temperature ranging from 50° C. to 300° C.; and
D) positioning the hot support element on the cosmetic product P at the access opening of the chamber of the mould; and
E) second cooling of the cosmetic product until solidification of the melted cosmetic product.

24. The process according to claim 21, further comprising steps of:
F) applying a layer of adhesive material on the support element; and
G) placing the cosmetic product P in a primary container.

25. The process according to claim 24, wherein the cosmetic product P is extracted from the mould before or after step F).
